(19) United States
(12) Patent Application Publication
(10) Pub. No.: US 2007/0088225 A1
(43) Pub. Date: Apr. 19, 2007

(54) End device

(76) Inventors: Takeshi Tanaka, Akisima (JP); Shunzo Yamashita, Musashino (JP); Goichi Ono, Soka (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/490,180

(22) Filed: Jul. 21, 2006

(30) Foreign Application Priority Data
Oct. 17, 2005 (JP) 2005-301126

(51) Int. Cl. A61B 5/02

(52) U.S. Cl. 600/503

(57) ABSTRACT

An end device attachable to the body and containing a heartbeat detector capable of low-power operation. The end device includes a light receiver element and current-to-voltage converter and an amplifier to amplify the output voltage from the current-to-voltage converter and a microcomputer; and the electrical current flowing in the light receiver element from which a specified current is subtracted, is input to the current-to-voltage converter, and the microcomputer CPU detects the heartbeat based on the signal from the amplifier.

### CROSS-REFERENCE TO RELATED APPLICATION

This application is related to U.S. application Ser. No. 11/208632 filed on Aug. 23, 2005, and U.S. application Ser. No. 11/210740 filed on Aug. 25, 2005, the disclosure of which is hereby incorporated by reference.

### CLAIM OF PRIORITY

The present invention claims priority from Japanese application JP 2005-301126 filed on Oct. 17, 2005, the content of which is hereby incorporated by reference into this application.

### FIELD OF THE INVENTION

The present invention relates to an end device including a pulse (heartbeat) detection function, and relates in particular to a wearable (attachable to a body) terminal.

### BACKGROUND OF THE INVENTION

Network systems (hereafter, sensor networks) are being developed in recent years that incorporate information processing devices for handling in real-time, different types of real-world information obtained by adding compact electronic circuits containing wireless communication functions in a sensor.

Sensor networks are made up of wireless networks comprised of multiple electronic circuits (hereafter called sensor nodes) including a wireless communication function, a sensor, and a power supply such as a battery, installed in the peripheral environment. It may therefore be crucial that these sensor nodes are maintenance-free over long periods, also capable of continually sending sensor data, and moreover possess a tiny outer profile. Development of extremely compact sensor nodes capable of being installed anywhere is therefore in progress.

One sensor node that attaches to the human arm is a tiny electronic circuit in a wrist-band shape including a wireless circuit, processor, sensor, and battery. The sensor node detects the number of heartbeats of a person from a heartbeat (or pulse) detector on the surface of the sensor node, and is capable of applications such as monitor the health status from remote locations by way of a communication network such as a LAN or the Internet. Making a sensor that is small and consumes little power is essential in order to permit long-term sensor node operation from a tiny battery.

One example of technology for low power consumption is intermittent sensor node operation as shown in JP-A No. 260291/2005. Main operations such as sensing and wireless communication end within a short time and so need only operate once every several seconds or every several minutes, so that power to the sensor, RF chip, and microcomputer can be shutoff at all other times to suppress power consumption and only made to operate at each preset time.

One heartbeat detector of the related art on the other hand, irradiates light onto the surface of the body, and then utilizes the change in intensity sustained by reflected light and scattered light obtained from the heartbeat within the irradiated portion of a vein, to detect the number of heartbeats from the received light signal. The example of the related art in JP-A No. 160641/2005, utilizes light at two different wavelengths to detect the heartbeat from the differential |signal (difference in signals) between the two wavelengths of received light. In the process when measuring the heartbeat, the intensity of DC components other the heartbeat signal component fluctuates irregularly due to body movements or the surrounding environment, and the intensity of the entire received light signal also fluctuates greatly according to those changes. In order to remove the DC components affected by fluctuations due to body movements and external light, a light source for two wavelengths is utilized including a wavelength whose light tends to easily reflect from the effect of blood flow, and a wavelength whose light mainly tends to reflect from the body surface without sustaining other effects. A stable heartbeat can then be detecting by obtaining the differential signal (difference in signals) between these two received light signals.

The technology in the JP-A No. 135330/2001 on the other hand performs subtraction compensation of a portion of the received light signal by utilizing an offset circuit. More specifically, the DC component within the received light signal is not needed for A/D conversion so installing an offset circuit allows compensating (or offsetting) the heartbeat signal components mainly within a range permitting A/D (analog-to-digital conversion, and that compensated (offset) signal is then input to the A/D converter section.

The technology in the JP-A No. 139862/2000 detects the heartbeat signal from the differential versus the reference voltage set beforehand. More specifically, the reference voltage is calculated beforehand according to the light intensity of the light source in order to reduce the effect mainly on the DC component in reflected light and scattered light whose intensity fluctuates greatly according to the light emission intensity of the light source. By then amplifying the differential between the received light signal and the reference voltage, unwanted intensity fluctuations within the received light signal are reduced.

### SUMMARY OF THE INVENTION

However, a small size and light weight are essential to allow attaching a sensor node with an internal heartbeat detector to a part of the body for long periods without causing discomfort. The internal or attached battery must therefore also be small. The power consumption is therefore limited due to the operating time.

The technology of the related art in JP-A No. 260291/2005 requires a long time for heartbeat detection and is therefore not suited for intermittent operation. Not only is a large electrical current utilized in the light source for this heartbeat detection device compared to other sensors but a long time is required for detection (sensing) so that operation consuming large amounts of power is long even operating intermittently. The power saving from this technology therefore does not rival the power saving effect of sensor nodes of the related art.

Moreover, an analog filter cannot be used during operation (hereafter intermittent operation) that shuts down the power to unnecessary circuits during times these circuits such as for sensing or wireless transmission are not being operated in order to reduce power consumption over time. Noise canceling in the technology of the related art utilized an analog filter. However the analog filter requires time to stabilize after power is turned on again. So during intermittent operation where power is repeatedly turned on and off, extra time is needed prior to the heartbeat detection operation so that power consumption increased.

The heartbeat detection device as shown in JP-A No. 160641/2005 on the other hand, requires a large size and greater power consumption when using multiple types of power supplies. This technology required a longer time than other sensors to accurately detect heartbeats so that the light source required increased electrical current. In other words, the overall power consumption increased drastically. In addition, the multiple types of internal light sources required installation space so that making this device small was impossible.

The technology of the related art in JP-A No. 135330/2001 compensates (offsets) the signal by an offset circuit installed in a state prior to input for A/D conversion. Saturation of the amplifier in that prestage is unavoidable. In this heartbeat detection method, most of the received light signal light is a DC component generated by the light of the light source reflected or scattered light on the body surface or by intrusion of external light such as sunlight. There is a limit to the A/D conversion resolution when inputting this signal as digital information so the A/D conversion accuracy of the heartbeat signal deteriorates. An offset circuit is installed prior (upstream) of A/D conversion in the technology of JP-A No. 135330/2001, however the signal input for A/D conversion must be amplified by an amplifier in a prestage. The amplifier gain must be raised in order to detect the heartbeat signal especially when the light emission intensity was lowered in order to reduce electrical current flow in the light source to lower power consumption. However the percentage of DC component in the received light then becomes large, and the amplifier then saturates due to irregular fluctuations in that (light emission) intensity.

In the technology in JP-A No. 139862/2000, the amplifier saturates due to fluctuations in the DC component. This technology utilizes a difference signal versus a reference voltage set beforehand according to the light emission intensity. However this intensity varies according to the usage environment and the actions of the person wearing the sensor node so that the DC component fluctuates irregularly, and leads to saturation of the amplifier since obtaining just the heartbeat signal alone is impossible.

This invention provides an end device attachable to a body, and includes a light emitter element, and a light receiver element to receive scattered light and reflected light from the light emitter element, and convert that light into an electrical current according to intensity of the received light; and a current-to-voltage converter circuit; and an amplifier circuit for amplifying the output voltage from the current-to-voltage converter circuit; and a microcomputer; and the electrical current flowing in the light receiver element from which a specified current is subtracted is input to the current-to-voltage converter circuit, and the microcomputer then detects the heartbeat based on the signal from the amplifier circuit. When the light emitter element emits light prior to detecting the heartbeat by the microcomputer in the terminal attached to the body, a specified quantity of electrical current is set based on the electrical current flow in the light receiver element that received the reflected and scattered light.

The microcomputer includes a first operating mode and a second operating mode. The first operating mode utilizes a higher frequency than the second operating mode. In the second operating mode, the microcomputer controls the light receiver element and loads the signal from the amplifier circuit. In the first operating mode, the microcomputer detects the heartbeat based on the signal from the amplifier circuit.

This invention is capable of detecting the heartbeat at the low power consumption required in end device attachable to a body.

### BRIEF DESCRIPTION OF THE DRAWINGS

FIG. 1 is a block diagram showing the structure of the sensor node contained within the heartbeat detection device;

FIG. 2 is a block diagram for showing the heartbeat detection device;

FIG. 3 is a drawing for showing the signal waveforms in the heartbeat detection process:

FIG. 4 is a block diagram for showing a typical structure of the I/V converter circuit;

FIG. 5 is a diagram for showing a typical structure of the variable current circuit;

FIG. 6 is a drawing for showing the operation flow in the heartbeat detection device;

FIG. 7 is a drawing showing an example of the external appearance of the heartbeat detection device containing the sensor node;

FIG. 8 is a flow chart for showing the embodiment of the sensor network system;

FIG. 9 is a flow chart showing the operation flow in the server, base station, and sensor node in the sensor network system; and

FIG. 10 is a drawing for showing the interrelation of power consumption with the sensor node operating states.

### DETAILED DESCRIPTION OF THE PREFERRED EMBODIMENTS

The embodiment of the end device (or heartbeat detection device) of this invention is described next while referring to the accompanying drawings.

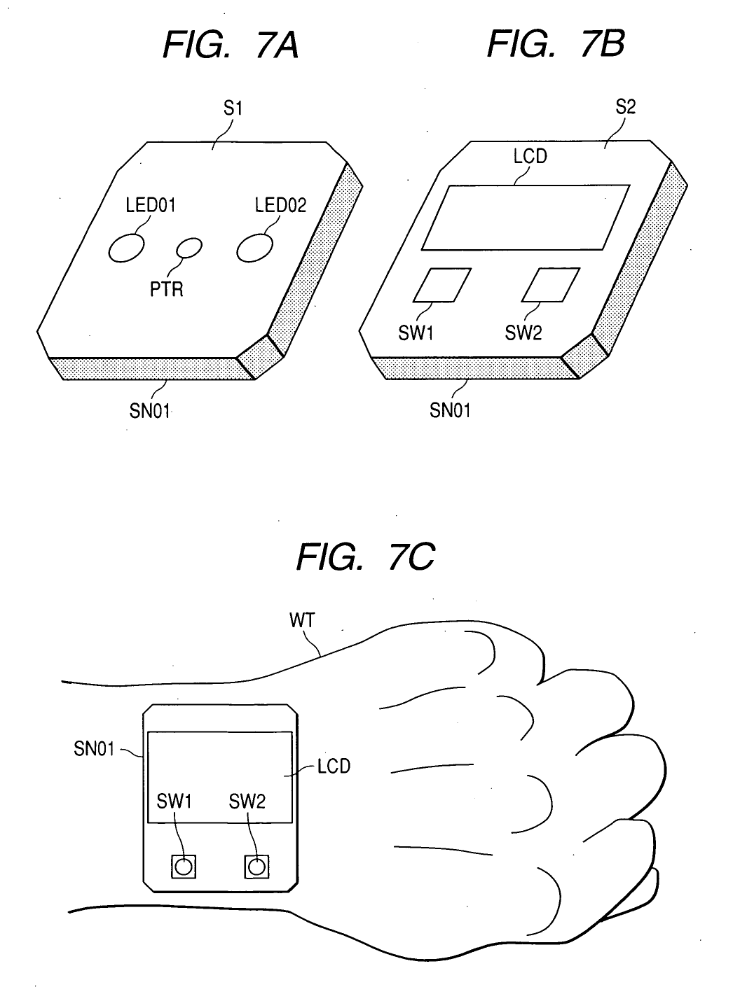

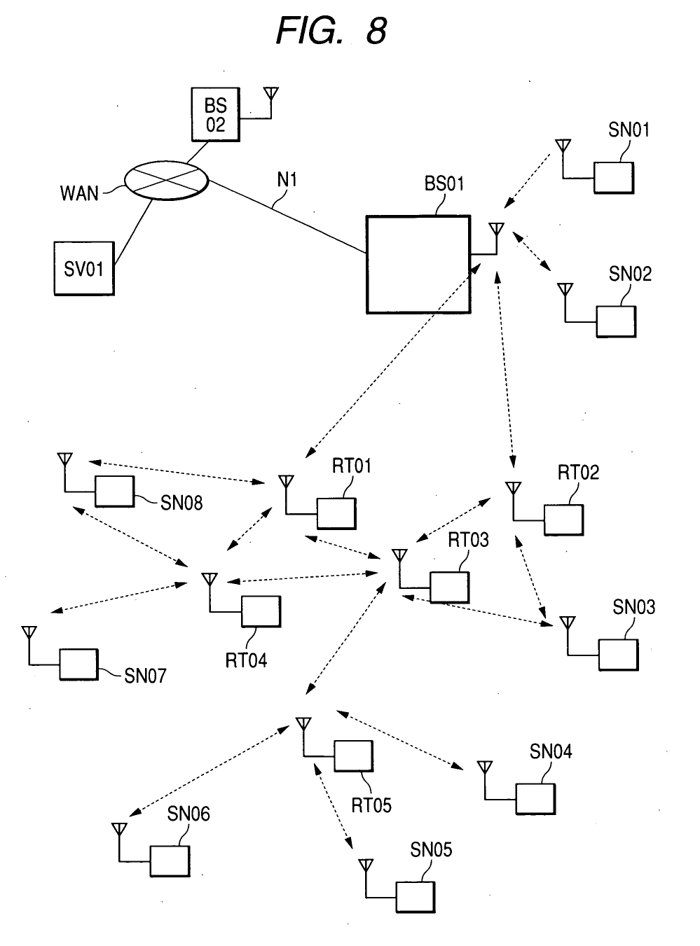

FIG. 7A through 7C show an outer view of the sensor node SN01 mounted in the heartbeat detection device of this invention. FIG. 7A shows the rear side of the sensor node SN1. FIG. 7B shows the front surface of the sensor node SN1. FIG. 7C shows the sensor node SN1 while attached to the arm WT of a person, when for example clamped by a wristband to the arm of a person. A phototransistor PTR in the heartbeat detection device senses the light emitted from the light emitter diode LED, and finds the pulse based on the change in intensity of the detected light. However as shown in FIG. 7A, preferably multiple light emitting diodes LED 1 and light emitting diodes LED 2 are installed along the periphery of the phototransistor PTR. Using multiple light emitting diodes allows irradiating a wider surface from the arm WT, and acquiring a signal spanning a wide range.

The surface (S1) containing the light emitting diodes (LED 1, LED2) and the phototransistor PTR is attached to the arm WT as shown in FIG. 7C. The surface (S1) containing the phototransistor PTR is attached to the arm WT at this time in a state making intrusion of external light difficult to make it less susceptible to fluctuations in the DC component caused by changes in the external light. The surface (S2) containing the display device LCD and external switches (SW1, SW2) is attached to the arm in a state that allows operating the external switches (SW1, SW2) and viewing the display device LCD. The display device LCD for example can be viewed while the number of heartbeats is being detected within the microcomputer CPU during heartbeat detection, and can display the heartbeat signal waveform. The display device LCD can also display the number of heartbeats detected after completing the detection operation, and display other sensing data. The display device LCD can also display the operation results (transmit end, failures, communication state, etc.) when sending and receiving these datum by wireless communication.

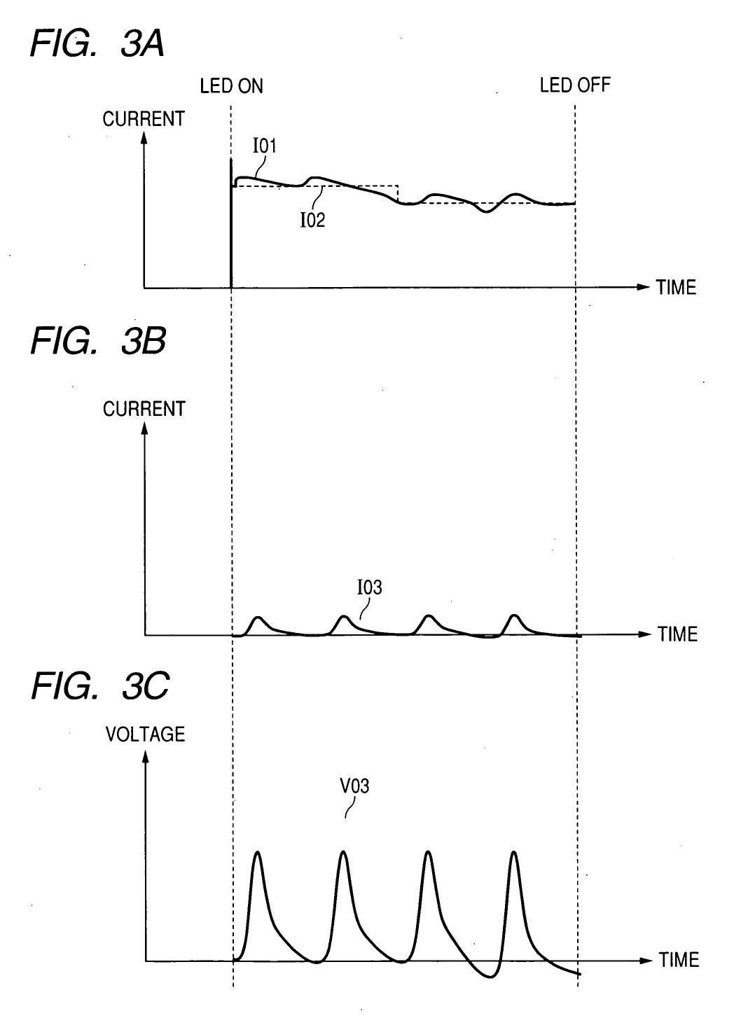

FIG. 3A through FIG. 3C show typical current and voltage signals obtained by the heartbeat detection device of the sensor node SN1. The attachment state of the sensor node to the arm as shown in FIG. 7, is the same as that for a wristwatch so that there is probably little psychological burden on the wearer. However, this device is characterized in that when the vein (blood vessel) density is low, then the amplitude of the signal showing the heartbeat within the entire signal detected by the phototransistor is slight compared for example to detecting the heartbeat from a section with a high vein (blood vessel) density. FIG. 3A is an example of a current signal waveform for the (electrical) current I01 detected from the phototransistor. As can be understood here, the DC component makes up most of the current I01 so that the gain of the amplifier will already be saturated even if that unchanged signal is amplified, and therefore the heartbeat component cannot be amplified.

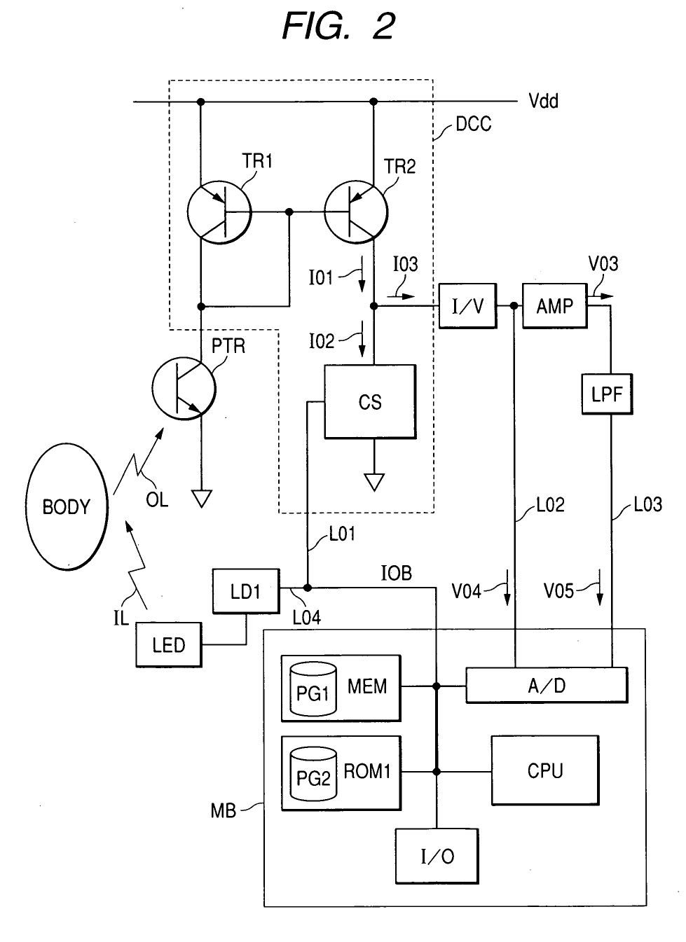

FIG. 2 is a circuit schematic showing the heartbeat detection device of this invention. The heartbeat detection device starts detecting the heartbeat signal from the body, when the detection operation starts. First of all, the light emitting diode LED irradiates an input light IL onto the body surface BODY (arm WT in the example in FIG. 7.). Infrared light is satisfactory for enlarging the effect from reflected and scattered light due to the input light IL in the vein (blood vessel) section. Most of the input light IL reflects from the surface of the skin but a portion is scattered or reflected in the veins (blood vessels) underneath the skin so that the intensity of the reflected-scattered light changes according to the blood flow. The change in intensity from the heartbeat (pulse) is large in body sections where there is a high vein (blood vessel) density such as the fingers because the heartbeat occupies a larger percentage of the reflected-scattered light OL so detection is easy. On the other hand, the change in intensity is small in sections such as the arm where the vein density is small due to the portion of the scattered-reflected light OL occupied by the heartbeat, so detection is difficult. The phototransistor PTR receives the scattered-reflected light OL. The phototransistor PTR converts the reflected light OL into electrical signals, and an equivalent value obtained via a current mirror circuit made up of transistors (TR1, TR2) flows in an electrical current 101. The waveform of the current signal I01 is shown in FIG. 3A.

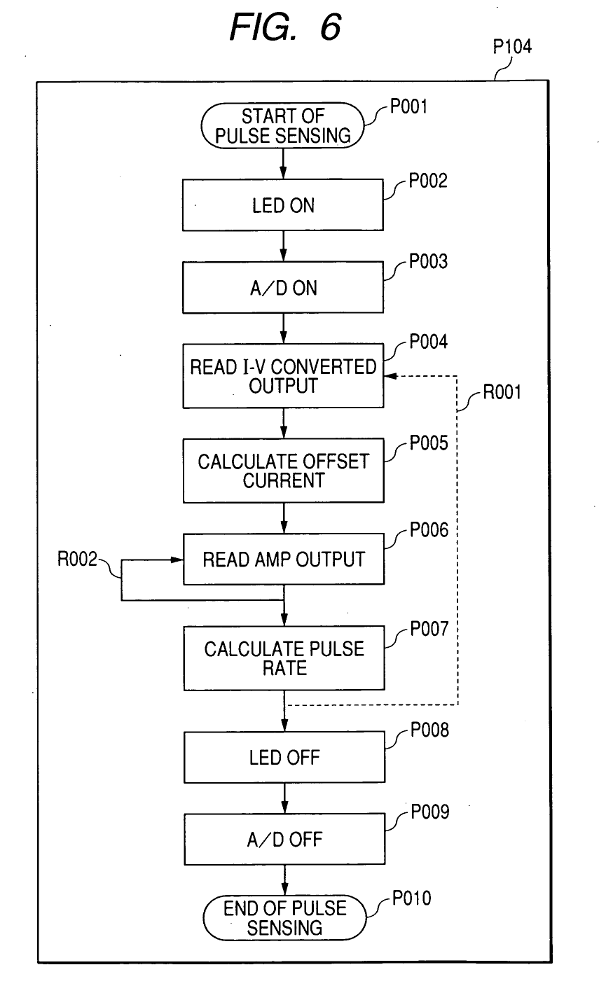

The operation is explained in detail while referring to FIG. 6. After attaching the sensor node SN1, the I/V converter circuit (I/V) converts the reflected light OL of the current signal at the start of sensing the heartbeat, into a voltage signal V04, and the voltage signal V04 is input from the signal line L02 to the main board MB. The A/D converter circuit (A/D) converts the voltage signal V04 from an analog signal to a digital signal, and inputs it to the microcomputer CPU. The DC component is then subtracted from the current signal I01 based on this voltage signal V04. In this embodiment, the quantity of electrical current corresponding to the input signal V04 is seen as comprising the DC component of the current signal I01. The reason for this is that the virtually all of the signal intensity is viewed as dominated by the DC component as shown in FIG. 3A. The microcomputer CPU sets the value of the CD component from the input signal V04, regulates the variable current circuit CS from the input-output device I/O via the control signal line L01, and the D/A converter circuit (D/A), and by generating a DC current in the variable current circuit CS that is equivalent to DC component of the received light signal, inputs a current signal 103 whose main component is the heartbeat signal, into the amplifier circuit AMP. More specifically, by making a canceling current equivalent to the DC component of the received light determined by the variable converter circuit CS flow in the signal line 102, the current 103 obtained by subtracting the current 102 as the DC component from the current 101 as the received signal, flows in the signal line 103 (FIG. 3B). The I/V converter circuit (I/V) converts the current signal I02 into a voltage signal and further amplifies it in the amplifier circuit AMP. The main component of the signal input to the amplifier circuit AMP is therefore the heartbeat signal, so that the signal is amplified within the waveform region of the amplifier circuit AMP and the heartbeat of the wearer (subject) can be accurately measured.

The voltage signal V03 amplified in the amplifier circuit AMP is processed in a low-pass filter LPF to cut the high-frequency noise, and after conversion to a digital signal in the A/D converter (A/D), is input to the microcomputer CPU. The microcomputer CPU processes the input signal V05 using processing recording in the programs (PG1, PG2) recorded in the memory MEM and the non-volatile memory ROM 1. A digital filter is applied to the input signal V05 for removing noise comprised of frequency components different from the heartbeat frequency (approximately 1 Hz). The number of heartbeats is then detected by calculating the peak from the signal still remaining after the digital filter processing.

Utilizing the digital filter to remove noise in this way allows high speed operation even if performing intermittent operation. If utilizing an analog filter then time is required for the filter to stabilize after the power is turned on, so that extra power is consumed during that time. In other words, a digital filter is more suited than an analog filter for intermittent operation that repeatedly turns the power off and on.

The heartbeat signal can be extracted by using a wavelength of only one light source by subtracting the DC component from the received light signal. Excess power consumption can for example be suppressed by decreasing the number of light sources, rather than by using methods that subtract the differential in received light signals while using multiple light sources. Moreover, less installation space is needed when using one type of light source so that the heartbeat detection device and sensor node can be made compact. This heartbeat detection device is ideal for use while attached for example to the arm of a person is ideal in terms of compactness and energy-saving.

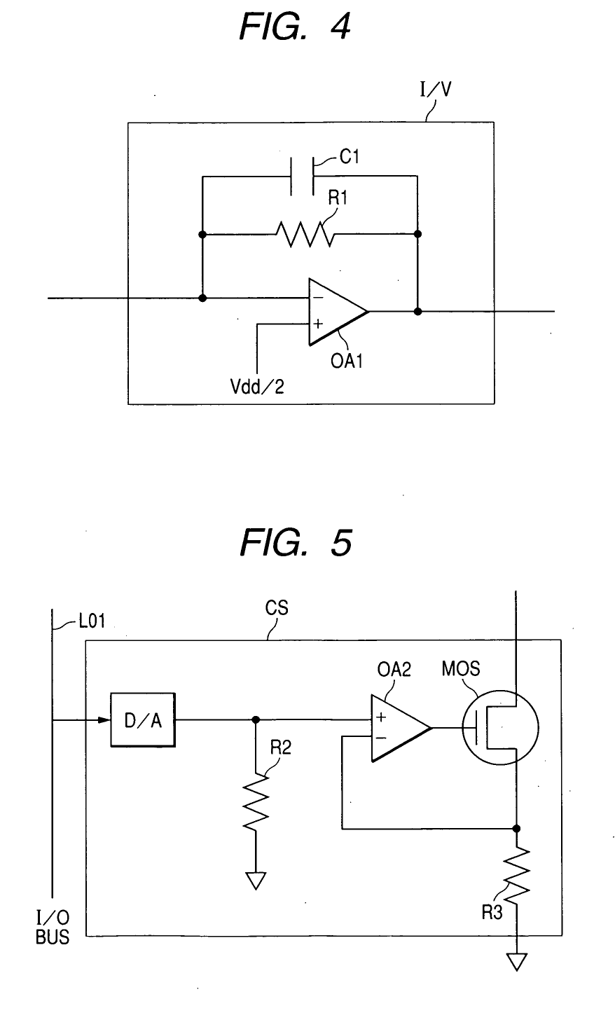

FIG. 4 is a schematic showing an example of the circuit structure of the I/V converter circuit (I/V). This circuit includes an operational amplifier OA1, a resistor R1, and a capacitor C1. A voltage Vdd/2 is applied for example as the reference voltage of the operational amplifier (hereafter “op amp”) OA1, and the output of op amp OA1 is fed back via the resistor R1 or the capacitor C1 versus this reference voltage. The advantage of a amplification from a simple circuit structure can therefore be achieved even in cases where the current signal I03 is negative (in other words, in cases where the current signal I01 is less than the current signal I02) by applying a value intermediate between the power supply voltage of op amp OA1 and the reference voltage of the heartbeat detection device, as the reference voltage.

FIG. 5 shows an example of a circuit diagram of the variable current circuit CS. This circuit includes a D/A converter circuit (D/A), an operational amplifier (hereafter “op amp”) OA2, a MOS transistor MOS, and the resistors R2, R3. The D/A converter circuit (D/A) converts the digital signal input from the control signal line L01 into an analog voltage signal. The op amp OA2, the MOStransistor (MOS), the resistors (R2, R3) change the gate voltage of the MOS transistor (MOS) according to the positive input to the op amp OA2 from the analog voltage signal, convert it to a current signal, and then output it. The voltage across the positive and negative inputs of the op amp OA2 is changed according to the ratio (value) between the resistors (R2, R3) and allow adjusting the current across the negative inputs of op amp OA2 from the MOA transistor (MOS).

FIG. 6 is a flowchart showing the operation flow for heartbeat detection in the heartbeat detection device. After attach the sensor node SN1, the microcomputer CPU first of all turns on the power (P002, P003) to the light emitting diode LED and the A/D converter circuit (A/D) when heartbeat detection start P001 is executed. Next, the I/V converter circuit (I/V) inputs the I/V converted received light signal V04 to the microcomputer CPU (P004). Most of this received light signal V04 is made up of the DC component so this value is set as the DC component value. By regulating the variable current circuit CS based on this value, a cancel current equal in value to the DC component is made to flow in the variable current circuit DC (P005). This remaining signal component is amplified in the amplifier circuit AMP and input to the microcomputer CPU (P006). This value is repeatedly taken and a waveform varying with changes in time is then formed (R002). The digital filter removes frequency components other than the required heartbeat signal from this time change waveform, and calculates the number of heartbeats by extracting the number of peaks from the remaining signal. The microcomputer CPU turns off the light emitting diode LED (P008) and the A/D converter circuit (A/D) (P009) when the number of heartbeats is detected, stops unnecessary power consumption, and terminates the heartbeat detection operation. (P010).

The operation (P004, P005) to calculate the cancel current from the received light signal need be performed only one time during the start of heartbeat detection. Moreover, if heartbeat detection is performed multiple times, then a quick response can be made to changes in the intensity of the DC component, a recalculation made at that time, and the current value of the variable current circuit CS also changed. Performing heartbeat detection multiple times allows the user to move around with the sensor node still attached, allows reducing the effect of DC component fluctuations while the body is moving, and improving the heartbeat detection accuracy.

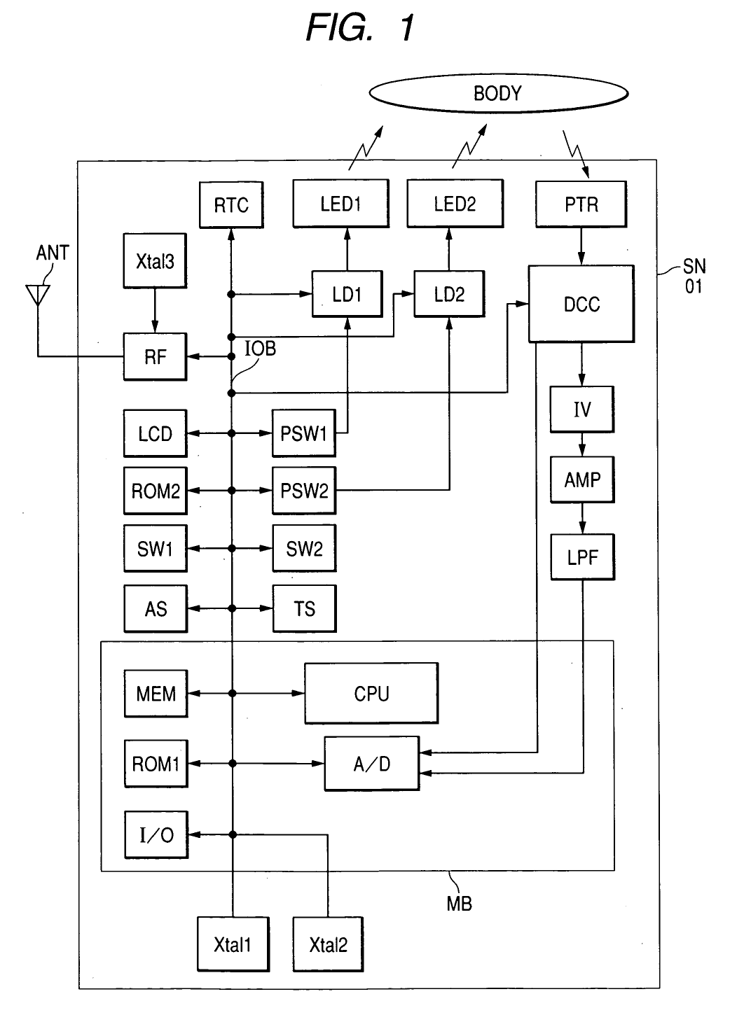

FIG. 1 shows a typical structure of the sensor node SN1 contained in the heartbeat detection device of this invention. The sensor node SN1 includes a main unit board MB (containing the microcomputer CPU, memory MEM, nonvolatile memory ROM1, input/output device I/O, A/D converter circuit (A/D)), a light emitting diode (LED1, LED2), a phototransistor PTR, a DC current cancel circuit (DCC), an amplifier circuit AMP, and a low-pass filter LPF.

Heartbeat detection is performed the same as previously described using FIG. 2 and FIG. 3. The microcomputer CPU controls the heartbeat detection. Multiple light emitting diodes (LED 1, LED2) are installed to emit light on the same wavelength as described in FIG. 7A. The microcomputer CPU controls the LED switches (PSW1, PSW2) via the input output bus IOB from the input/output device I/O to make the light emitting diodes (LED1, LED2) emit light. Therefore in the circuit structure in FIG. 1, the reflected light-scattered light OL from the incoming light from the two light emitting diodes is received by the photodiode PTR.

Applying the present invention renders the effect of lowering the power consumption of the light emitting diodes (LED1, LED2) since the gain of the amplifier circuit AMP can be increased while avoiding saturation of the amplifier AMP by the DC component in the received light signal. Raising the light emission intensity of the light emitting diodes (LED1, LED2) was considered as a way to increase the change in reflected light-scattered light OL occurring due to the heartbeat. However the power consumption then increases so this method is not suited for wrist type sensor nodes SN1 that will be operated for long periods of time from limited power sources such as button batteries. In this invention however, the gain of the amplifier circuit AMP can be raised and the input to the microcomputer CPU amplified to the required intensity even with a weak heartbeat signal so that the current in the light emitting diodes (LED1, LED2) can be lowered. What should be noticed here is that the light emitting diodes (LED1, LED2) make up a large share of the actual power consumed in the wrist-type sensor node SN01 so that power consumption in the sensor node SN01 can be drastically reduced.

The light emitting diodes (LED1, LED2) power consumption can also be reduced by using the microcomputer CPU to limit the light emission intensity of light emitting diodes (LED1, LED2). The microcomputer CPU for example can monitor the intensity of the heartbeat signals during input, and compare them using the input signal strength established in programs (PG1, PG2) as a reference. The microcomputer CPU can then lower the light emission intensity when the input signal is strong, and raise the light emission intensity when the input signal is weak. More specifically, the microcomputer CPU sends a control signal from the input/output device I/O via the I/O bus (I/OB), to regulate the LED drivers (LD1, LD2), and adjust the light intensity of the light emitting diodes (LED1, LED2). This operation maintains the required light emission intensity while operating the light emitting diodes (LED1, LED2) so as to suppress power consumption.

The detected heartbeat count and other sensing data, and operation information for the sensor node SN1 is sent as a wireless signal from a wireless chip RF connected to an antenna ANT. The operation information includes device connection information such as other adjacent nodes and wireless communication quality, a transmit quality history (successful transmissions—number of failures, etc.) up to the present, battery information, and hardware-software versions, etc. This information is required for managing sensor networks made up of sensor nodes, and for optimizing sensor node installation locations, etc.

Sensors other than heartbeat detection devices can be installed and operated in the sensor node SN01. A velocity sensor AS for detecting human movement and temperature sensor TS for detecting body temperature and human skin surface temperature can for example be installed. A velocity sensor mounted in the sensor node SN01 can detect movement of a person by way of the velocity sensor AS value and in this way allows estimating the operating state and the actions of the user wearing the sensor node. A reliability index of the number of heartbeats detected by the heartbeat detection device can in this way be contrived. More specifically, a heartbeat detection measurement made while a person is very active gives a heartbeat count with low accuracy and low reliability because the received light signal contains scattered light and reflected light that is different from when the body is in a relaxed state, because of the muscle movement within the body. By referring to the velocity sensor AS value, one can determine beforehand whether the detected heartbeat count is a reliable numerical value. If the sensor node contains a temperature sensor TS, then the health condition of the wearer can be known in detail by measuring to find a numerical value along with the temperature information or heartbeat information.

This same information can also be displayed on a display device LCD to inform the user of the numerical (measurement) value. Information detected by the sensor node SN01 can be sent not only to the administrator by wireless, by the contents of that information can only be reported on the spot to the user himself. Information showing information identifying the connected network or information showing the wireless (radio) frequency can also be displayed and notification given. During intermittent operation, the current time can also be displayed even in a standby state. A history (log) of the detected information and sensor node information can be store in the external non-volatile memory ROM2. The wireless communication might sometimes be interrupted due to effects from absorption or reflection of transmitted radio waves due to the surrounding environment when the user carrying the sensor node SN01 is moving or in action. The transmit data cannot be sent to the transmit destination in this state, however the sensing data that was acquired and information on the time the data was acquired can be stored in the non-volatile memory ROM2, and then sent the next time that communication is possible. Damage to sensing data due to changes in the wireless communication status can therefore be prevented, and a stable supply of information ensured.

The sensor node SN1 includes an external clock (Xtal1, Xtal2) for making inputs to the microcomputer CPU and, an external clock (Xtal3) for making inputs to the wireless chip. The microcomputer CPU and wireless chip operate based on the time from these external clocks.

To reduce power consumption over time and allow long term operation, the sensor node SN1 operates by turning the power off during processing such as sensing and wireless communication in circuits (wireless chip RF, microcomputer CPU, clock Ttal1 to Xtal3, etc.) and turning the power back on again when needed (hereafter called “intermittent operation”. Intermittent operation is performed by operating at times reset by the programs (PG1, PG2) or times stored in the external non-volatile memory ROM2 for making changes after startup. Operating states such as heartbeat detection or wireless sending/receiving start at each predetermined time, and at all other times the power to the light emitting diodes (LED1, LED2) is turned off by the power supply switches (PSW1, PSW2), and unnecessary power consumption can then be suppressed by turning off power to all other unnecessary circuits except the microcomputer CPU, wireless chip RF, external clocks (Xtal1, Xtal2, Xtal3) and real-time clock RTC. During the standby period, the real-time clock RTC counts the intermittent operating time determined by the programs (PG1, PG2), and when that predetermined time elapses, again turns on the power to perform preset operations such as, detecting heartbeats and wireless communication.

The sensor node SN1 contains switches (SW1, SW2) that are externally operated and perform interrupt (break-in) operation in the microcomputer CPU as set by the programs (PG1, PG2). The external switches (SW1, SW2) can be operated to show settings for the sensor node intermittent operation period or wireless (radio) status on the display device LCD. When these values must be changed, the switches (SW1, SW2) can be operated to make the changes while referring to the information on the display device LCD, the changes then stored in the external non-volatile memory ROM2, and those changed settings then used in the next operations.

The power to the display device LCD can also be turned off at all other times than during heartbeat detection or wireless communication in order to reduce power consumption. The current time can also be displayed. The time displayed then can be obtained via wireless communication and stored in the external non-volatile memory ROM2. Among other means, the user can make changes manually while checking them with the external switches (SW1, SW2), and those changes may also be stored in the same way in the external non-volatile memory ROM2.

FIG. 8 is drawings showing the sensor network system containing the sensor nodes. The base station is an electronic circuit including a means for connecting to wide area communication networks, storage devices, and processing devices for processing such as wireless communication functions and recording and sending data from the sensor node. The router is a electronic circuit for receiving data sent from other sensor nodes, and performing relay operations (hereafter called, “routing”) for sending data destined for delivery to the base station and other sensor nodes, to the nearest base station, router or other destination sensor nodes. The sensor node relays data, and may sometimes perform the same functions as the router. The sensor node (SN01-08) is alternately connected by wireless communication to the base station BS01, or the routers (RT01-05).

The routers (RT01-05) perform routing of information from the sensor nodes (SN01 to 08), the routers (RT01 to 08) and base station BS01, to the respective transmit destinations for that information. The routers (RT01-05) can send path search data for discovering ahead of time, the most efficient transmit path along which to send the information. Storing this path search data (hereafter called routing table) allows performing subsequent routing with good efficiency. Routing can also be performed based on preset programs. In that case, identification numbers capable of expressing the connection relation are attached to the routers (RT01-05), sensor nodes (SN01 to 08), and base station BS01, and data routing then performed based on those identification numbers. Many routers or sensor nodes (such as SN01 to 08) possessing the same functions as routers, can be installed in locations where wireless communication between the base station BS01 and sensor nodes (SN01 to 08) was difficult due to the distance and RF interference and sensing data can in this way be collected from a wide environment. Moreover when the sensor nodes (SN01 to 08) are attached to a person, and there is wireless (radio) communication with the base station BS1, the range that the sensing data can be transmitted is limited to the propagation range of radio waves from the base station BS01 and is therefore not suited for use when the user is moving. However, by installing many routers (RT-01 to -05) in range where the radio waves can mutually reach each other, then the user wearing the sensor nodes (SN01 to 08) can send sensing data while being active in a wider range.

The router and the base station BS01 are connected by wireless line N1 to a wide area communication network WAN1 such as LAN or the Internet. Other base stations BS02 and amassed data, and the base stations (BS01, BS02) are connected via the wide area communication network WAN1 to a server SV01 for sending control information. Users making use of the sensor network system, or applications operating to fulfill various service objectives are connected to the server SV01 from terminal connected to the wide area communication network WAN, and acquire information such as sensing data from sensor networks by communicating as needed.

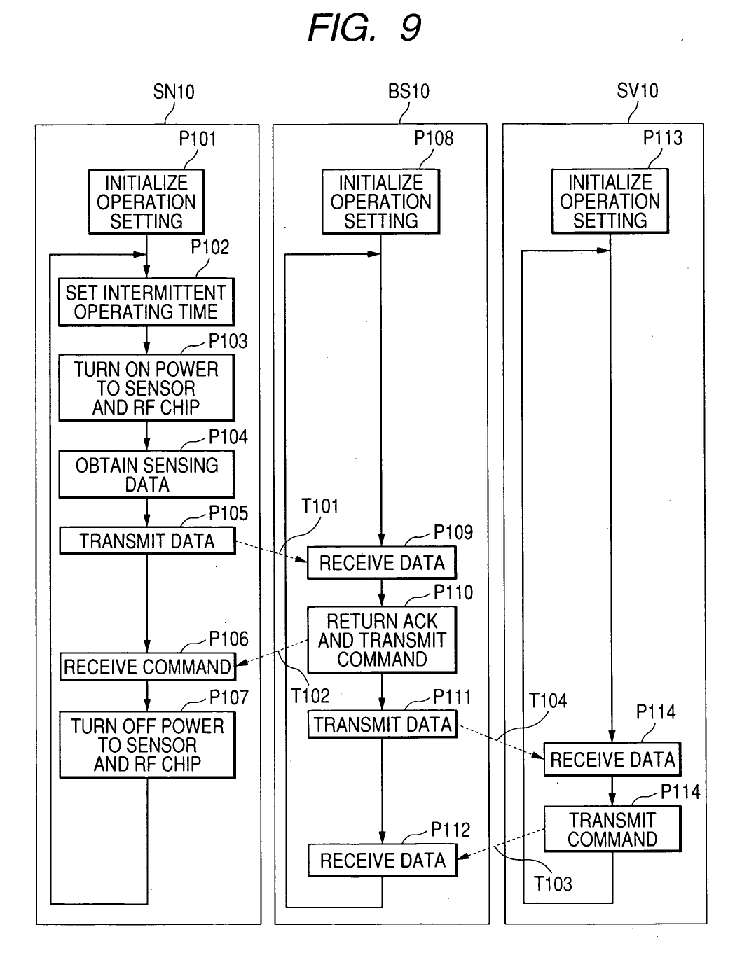

FIG. 9 is a flowchart showing sensor network system made up of a sensor node SN10, a base station BS10 and a server SV10. When the sensor node SN01 starts, the wireless settings and operation states such as wireless transmission power and wireless channels are reset, and participation requests are sent to the sensor networks comprising the base station BS10. When the base station BS01 receives this participation request, the base station BS01 sends a participation completion notification, the sensor node SN10 receives this so that sensing data can now be sent to the base station BS01, and intermittent operation starts (P101). The microcomputer CPU then starts operating, and sets the intermittent operation time (P102) which is the time from turning power off after sensing and wireless communication was performed, until operation restarts. The microcomputer CPU sets that time here by referring to the values stored in the external non-volatile memory ROM2. These reset (initialization) values are stored in the programs (PG1, PG2) and can be changed by commands sent from the base station BS01 or by operating the external switches (SW1, SW2). After setting the intermittent operation times, the power to the sensor and wireless chip is turned on (P103). Sensing operation is performed via heartbeat detection and other sensors, and sensing data is acquired (P104). Information acquired from the sensing operation is sent by wireless communication to the base station BS10 (P105). However if the base station BS10 and sensor node SN10 are at a distance farther than the radio waves can propagate (reach), then the data can be sent by relaying it through routers. The sensor node SN10 sets to receive standby after transmitting data (T101), and when it receives a data receive request (hereafter called Ack) and an operation execution request (hereafter called a command) (T102), commences receive processing to analyze and execute the received command and process the received command (P106). However, data retransmission or receive standby is terminated, if the wireless communication status deteriorates after the sensor node SN10 sends data to the base station BS10, or there is operational interference at the base station BS10 so that the Ack or commands cannot be received. The retransmit count for transmit data and the maximum time for receive standby (hereafter called receive timeout time) are set in the sensor node SN10. If no Ack reply is received from the base station BS10 or the router, then the transmit data is resent for the number of retransmit times (count) that were set. Command receive standby is performed within the receive time-out time after receiving the Ack command. The retransmit count for the transmit data and the receive time-out time are recorded in the programs (PG01, PG02). Power to the sensor, the wireless chip, and the microcomputer CPU (P107) after terminating the command receive processing (P106). The operation is then in standby to suppress unnecessary power consumption until the time set for intermittent operation (P102) elapses. After that time elapses, the operation (P102 to 107) repeats again from the time set for intermittent operation (P102).

The base station BS10 sets to wireless communication standby after resetting the operation settings (P108) and can then receive wireless transmissions from the node SN10 (T101) After starting, the base station BS10 accepts participation requests from the sensor node SN10 sending sensing data to base station BS10, to-participate in the sensor network. After receiving this participation request, the base station BS10 assigns identification numbers to the sensor node SN10, and identifies multiple sensor nodes. The base station BS10 performs data receive processing (P109) such as identifying the data or the sensor node of the transmit source, when it receives data such as sensor node SN10 sensing data, and sends an Ack. (T102) in reply. The base station BS10 transmits commands (T102) at this time when there are transmit commands in the waiting list (hereafter called the transmit queue) to send to the SN10. The base station BS10 attaches sensor node information (as transmit source), data information, and acquisition time information to the sensing information received from the sensor node SN10, and transmits it to the server SV10 (P111). The base station BS10 is usually in communication standby, awaiting messages from the server SV10. When the base station BS10 receives a data transmission (T103) from the server SV10, it processes the received command, to analyze the received data and to send transmit data for the sensor node to the transmit queue (P112).

After starting, and resetting (initializing) the operation settings (P113), the server SV10 is in standby state for communications (messages) sent to the SV10 from multiple base stations, and accepts data transmissions from the base station BS10 (T104). Preset replies and control information to the sensor network system from the user or administrator are transmitted to the appropriate base station (P114). The terminal connected to the network accesses the server SV10, and acquires information from the network system per the user, or transmits control information.

To lower the power consumption in the sensor node SN1 even further, the clock of the microcomputer CPU may be changed at the processing stage when performing heartbeat detection. Referring to the flowchart in FIG. 6, when heartbeat detection starts (P001), the microcomputer CPU operates in a low-clock state. The operation from steps P002 to P007 is then executed in a low-clock operating state. The operation from setting a DC component from the value of the received light signal of phototransistor PTR, to input of a heartbeat signal minus the DC component differential mainly requires about 15 seconds (changeable from several to several dozen seconds). However the processing load on the microcomputer CPU is small so processing can be performed in low-clock operation. Using the sensor node SN01 of FIG. 1 as an example, when this operation is performed in a low-clock state, the power consumption is approximately 1 mA which is drastically lower than high-clock operation.

After acquiring the heartbeat signal waveform, the microcomputer CPU sets to a high-clock operating state, and the operation to calculate the heartbeat (P007) is performed with the microcomputer CPU at a high clock operating state. This processing applies a load on the microcomputer CPU and high-speed operation is required, so that taking the sensor node SN01 in FIG. 1 as an example so that the power consumption is large at approximately 6 mA. However this high-clock operation at high power consumption can be suppressed to a minimum, by limiting the calculation time to a short time of approximately several milliseconds.

The light emitting diode LED is turned off and the A/D converter circuit (A/D) is turned off, and power to unnecessary circuits is turned off when the number of heartbeats count is detected (calculated) and the heartbeat detection operation then terminates.

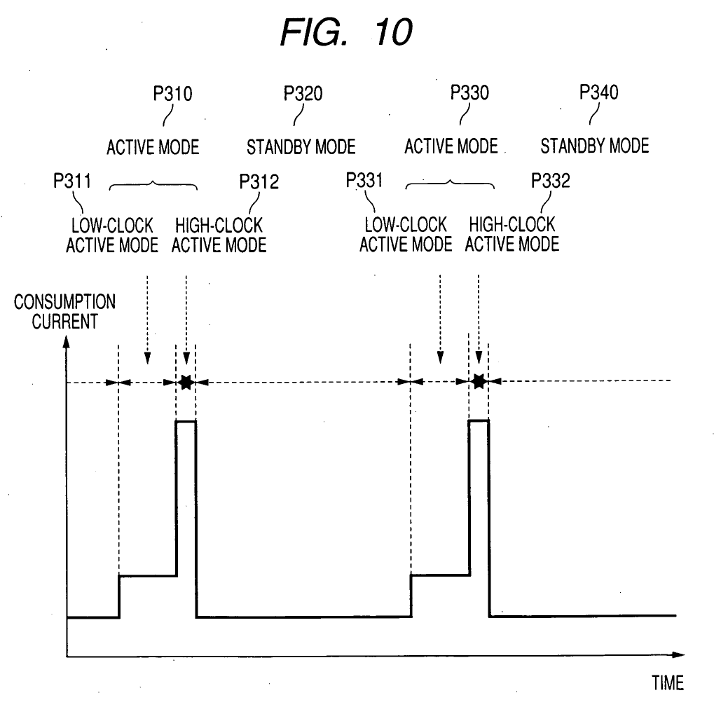

FIG. 10 shows the changes over time in current consumption in the sensor node during intermittent operation including heartbeat detection by varying the clock (as described above). Current consumption in the sensor node by the real time clock RTC and other circuits is smallest when in the standby state (P320, P340). The larger the percentage of time taken up by the standby state from the total time, the more the average power consumption over time can be lowered. Lengthening the operating time on limited battery (power) can be achieved by extending the intermittent operation time.

Electrical current consumption increases during the operating state (P310, P330) because the sensor, the wireless chip RF, and the microcomputer CPU are operating. The microcomputer CPU does not need to operate at high speed in the period where the signal waveform was acquired after the microcomputer CPU subtracts the canceling current from the light signal received from the phototransistor PTR. The microcomputer CPU therefore operates in a low-clock state (P311, P331) and so the power consumption in the microcomputer CPU can be limited compared to when in a high-clock state. The time required here is from several to several dozen seconds. If using the sensor node SN01 in FIG. 1, then the current is about 10 mA. In subsequent periods, the microcomputer CPU operates in a high-clock state, and performs processing such as digital filter processing, detecting the number of heartbeats, and wireless communication (P312, P332). The power consumption in the microcomputer is at the maximum here but the calculation time and wireless communication time is short so that the overall percentage of time required can be kept to a minimum. The current consumed during this time is approximately several milliamps. If using the sensor node SN01 in FIG. 1, then a current of about 60 mA is required. In other words, the overall current required for the operating state can be lowered. Afterwards, the process of turning off power to the sensor, the wireless RF chip, and the microcomputer CPU and then setting again to a standby state (P320, P330) is repeated.

What is claimed is:

1. An end device attachable to the body comprising:
a light emitter element;
a light receiver element to receive scattered light and reflected light from the light emitter element, and convert that light into an electrical current according to intensity of the received light;
a current-to-voltage converter circuit;
an amplifier circuit for amplifying the output voltage from the current-to-voltage converter circuit; and
a microcomputer,
wherein the current-to-voltage converter circuit is input with an electrical current flowing in the light receiver element from which a specified current is subtracted, and
the microcomputer then detects the heartbeat based on the signal from the amplifier circuit.

2. An end device according to claim 1, wherein a specified amount of current is set based on the amount of current flowing when the light receiver element receives scattered light and reflected light emitted from the light emitter element, before the operation to detect the heartbeat by a microcomputer in a terminal attached to the body.

3. An end device according to claim 2, wherein a specified amount of current is rewritten based on the amount of current flowing when the light receiver element receives scattered light and reflected light emitted from the light emitter element, during the heartbeat detection operation by the microcomputer in a terminal attached to the body.

4. An end device according to claim 1, including a wristband, wherein the end device is attachable to the arm of a human body by the wristband.

5. An end device according to claim 1, wherein
the microcomputer includes a first operating mode and a second operating mode,
the first operating mode operates at a higher frequency than the second operating mode, and
the microcomputer operates the second operating mode for controlling the light emitter element and reading the signal from the amplifier circuit, and operates the first operating mode to detect the heartbeat based on the signal from the amplifier circuit.

6. An end device according to claim 1, wherein
the light emitting element includes multiple light emitting sources, and
the multiple light emitting sources are all capable of emitting infrared light on the same wavelength.

7. An end device according to claim 1, including a wireless communication device, wherein the wireless communication device transfers the detected heartbeat information to the server.
